(19) United States
(12) Patent Application Publication
(10) Pub. No.: US 2016/0213884 A1
(43) Pub. Date: Jul. 28, 2016

(54) ADAPTIVE CATHETER CONTROL FOR PLANAR USER INTERFACE

(71) Applicant: Hansen Medical, Inc., Mountain View, CA (US)

(72) Inventor: June Park, San Jose, CA (US)

(21) Appl. No.: 15/007,881

(22) Filed: Jan. 27, 2016

Related U.S. Application Data
(60) Provisional application No. 62/108,210, filed on Jan. 27, 2015.

(51) Int. Cl. A61M 25/01; A61B 34/00; A61B 34/30; A61B 90/00

(52) U.S. Cl. CPC A61M 25/0133; A61B 90/37; A61B 34/25; A61B 34/30; A61B 2090/376; A61B 2034/301; A61M 2025/0166

(57) ABSTRACT

A method for manipulating a catheter within a lumen of a body may involve providing a manipulatable catheter system, including a catheter and a controller coupled with the catheter. The method may further involve: displaying an image of at least a distal portion of the catheter on a video display; receiving, via the controller, a user input directing the distal portion of the catheter to articulate; determining a relationship between an articulation plane of the distal portion of the catheter and a viewing plane of the image on the video display; automatically adjusting the catheter, using the controller, to move the articulation plane of the distal portion closer to parallel with the viewing plane of the image, based on the determined relationship; and articulating the distal portion of the catheter, using the controller, based on the user input.

### CROSS-REFERENCE TO RELATED APPLICATIONS

This application claims priority to U.S. Provisional Application No. 62/108,210, entitled “Adaptive Catheter Control for Planar User Interface,” filed Jan. 27, 2015, which is herein incorporated by reference in its entirety.

### BACKGROUND

Steerable catheters facilitate navigation in tortuous anatomy. Robotic manipulation of such catheters brings precision and accuracy to catheterized procedures. Despite advances in manipulation, physicians still rely on fluoroscopic imaging for visual feedback. Due to its inherently planar nature, fluoroscopy often fails to provide substantial information regarding the depth of an object shown in its image, which is an important piece of information in catheter manipulation. Without the depth cue, physicians often struggle to determine the orientation of the catheter, because it is unclear whether the tip of the device is pointing into or out of the screen. This affects the quality of a procedure as well as its duration.

### SUMMARY

Accordingly, there is a need for improved catheter manipulation systems and methods. For example, there is a need for systems and methods that improve manipulation of catheters within body lumens when catheters are guided by physicians using 2D imaging systems. There is a need for enhanced instinctiveness in catheter and other medical device manipulation. Various aspects of the present disclosure address one or more such needs.

One aspect of the disclosure is directed to a method for manipulating a catheter within a lumen of a body. Such a method may be performed, at least in part, with a manipulatable catheter system. The manipulatable catheter system may include a catheter having a proximal end, a distal end, an articulable distal portion, and a sensor disposed along the distal portion. The manipulatable catheter system may further include a controller coupled with the catheter proximal end, wherein the controller controls articulation of the distal portion of the catheter. The method for manipulating the catheter within the lumen of the body may include: displaying an image of at least the distal portion of the catheter on a video display; receiving, via the controller, a user input directing the distal portion of the catheter to articulate in an articulation direction; determining a relationship between an articulation plane of the distal portion of the catheter and a viewing plane of the image on the video display; automatically adjusting the catheter, using the controller, to move the articulation plane of the distal portion closer to parallel with the viewing plane of the image, based on the determined relationship; and articulating the distal portion of the catheter in the articulation direction, using the controller, based on the user input.

In some embodiments, determining the relationship may include determining a difference in orientation between the articulation plane and the viewing plane. In certain embodiments, adjusting the catheter may include aligning the articulation plane with the viewing plane so that the two planes are parallel. In some embodiments, adjusting the catheter may include rotating the catheter. In some embodiments, adjusting the catheter may include adjusting the articulation direction to align the articulation plane with the viewing plane. In some embodiments, adjusting the catheter may include adjusting the articulation direction to align the articulation plane with the viewing plane.

In some embodiments, the controller may include a left-articulation user input member and a right-articulation user input member, and the articulation direction may include a left direction or a right direction. In some embodiments, the image may include a fluoroscopic image.

In some embodiments, the method may further include determining an amount of adjustment before adjusting the catheter. In some embodiments, determining the amount of adjustment may include: determining a coordinate system, determining the viewing plane within the coordinate system, and determining the articulation plane within the coordinate system. In some embodiments, the method may further include: receiving an additional user input instructing the controller to allow the distal portion to articulate in the articulation plane without adjusting the catheter; and articulating the distal portion of the catheter as instructed by the additional user input.

In another aspect of the disclosure, a method for manipulating a catheter within a lumen of a body is provided. The method may be performed, at least in part, with a manipulatable catheter system. The system may include: a catheter having a proximal end, a distal end, an articulable distal portion, and a sensor disposed along the distal portion; and a controller coupled with the catheter proximal end. In various embodiments, the controller controls articulation of the distal portion of the catheter. The method may include the steps of: displaying a representation of at least the distal portion of the catheter on a video display that defines a viewing plane; receiving, via the controller, a user input directing the distal portion of the catheter to articulate in a left direction or a right direction, relative to the representation on the video display; automatically adjusting the catheter to align an articulation plane of the distal portion of the catheter with the viewing plane; and articulating the distal portion of the catheter to the left or right, according to the user input, within the articulation plane.

In some embodiments, adjusting the catheter may include aligning the articulation plane with the viewing plane so that the two planes are parallel. In some embodiments, adjusting the catheter may include rotating the catheter. In some embodiments, adjusting the catheter may include adjusting the articulation direction to align the articulation plane with the viewing plane. In some embodiments, the controller may include a left-articulation user input member and a right-articulation user input member configured to articulate the distal portion of the catheter to the left and the right, respectively, within the articulation plane. In some embodiments, the representation on the video display may include a fluoroscopic image.

In some embodiments, the method may further include determining an amount of adjustment before adjusting the catheter. In some embodiments, determining the amount of adjustment includes: determining a coordinate system, determining the viewing plane within the coordinate system, and determining the articulation plane within the coordinate system. In some embodiments, the method further includes: receiving an additional user input instructing the controller to allow the distal portion to articulate in the articulation plane without adjusting the catheter, and articulating the distal portion of the catheter as instructed by the additional user input.

In another aspect, a method for manipulating a catheter within a lumen of a body is provided. The method may include providing a manipulatable catheter system. The system may include: a catheter having a proximal end, a distal end, an articulable distal portion, and a sensor disposed along the distal portion; and a controller coupled with the catheter proximal end, wherein the controller controls articulation of the distal portion of the catheter. The method may further include: displaying a representation of at least the distal portion of the catheter on a video display that defines a viewing plane; receiving, via the controller, a user input directing the distal portion of the catheter to articulate in a left direction or a right direction, relative to the representation on the video display; automatically adjusting the catheter to align an articulation plane of the distal portion of the catheter with the viewing plane; and articulating the distal portion of the catheter to the left or right, according to the user input, within the articulation plane.

In some embodiments, adjusting the catheter may include aligning the articulation plane with the viewing plane so that the two planes are parallel. In some embodiments, adjusting the catheter may include rotating the catheter. In some embodiments, the image includes a fluoroscopic image. In some embodiments, the method may further include determining an amount of adjustment before adjusting the catheter. In some embodiments, determining the amount of adjustment involves: determining a coordinate system, determining the viewing plane within the coordinate system, and determining the articulation plane within the coordinate system.

In another aspect, a system for manipulating a catheter within a lumen of a human or animal subject is provided. The system may include: a catheter having a proximal end, a distal end, an articulable distal portion configured to articulate in three dimensions, and a sensor disposed along the distal portion; a controller coupled with the catheter proximal end to bend the distal portion; and a processor coupled with the controller and configured to execute instructions to perform a method. The method performed by the processor may include: determining a coordinate system; determining a viewing plane within the coordinate system, wherein the viewing plane is defined by an image of the distal portion of the catheter on a video display; determining an articulation plane of the distal end of the catheter; receiving a user input directing the distal portion of the catheter to articulate in a direction; and automatically adjusting the catheter to align the articulation plane with the viewing plane.

In some embodiments, the user input may include an instruction to articulate the distal portion of the catheter in a left direction or a right direction. In some embodiments, the processor may be further configured to generate an articulation signal to cause an actuator to articulate the distal portion of the catheter according to the user input. In some embodiments, adjusting the catheter may include rotating the catheter so that the articulation plane is parallel with the viewing plane.

These and other aspects and embodiments are described in further detail below, in reference to the attached drawings.

### BRIEF DESCRIPTION OF THE DRAWINGS

While the claims are not limited to the illustrated embodiments, an appreciation of various aspects is best gained through a discussion of various examples thereof. Exemplary illustrations are described in detail herein by referring to the following drawings:

FIG. 1 is a schematic diagram of a system for controlling an articulable device, according to one embodiment;

FIG. 2 is a perspective view of a control console of a robotic catheter system, according to one embodiment;

FIG. 3A is a side view of a distal portion of an articulable catheter, according to one embodiment;

FIG. 3B is an enlarged, side view of a portion of the distal portion of FIG. 3A;

FIG. 4 is a front view of an image display, illustrating a distal portion of an articulable catheter and its articulation plane, according to one embodiment;

FIG. 5A is a side view of a distal portion of an articulable catheter, illustrating its articulation plane, according to one embodiment;

FIG. 5B is a side view of the distal portion of FIG. 5A, after the catheter has been adjusted to align an articulation plane with a viewing plane, according to one embodiment;

FIG. 5C is a side view of the distal portion of FIG. 5B, with the distal portion articulated to the left, in response to a user input, according to one embodiment;

FIGS. 6A-6C are analogous to those of FIGS. 5A-5C but illustrate the distal portion of the catheter articulating to the right, according to one embodiment;

FIG. 7 is a front view of an image display, illustrating a distal portion of an articulable catheter and its articulation plane, including a superimposed coordinate system, according to one embodiment;

FIG. 8 is a perspective view of a distal portion of an articulable catheter, illustrating superimposed equations and markings; and

FIG. 9 is a flow diagram illustrating a method for articulating an articulable catheter or other device, according to one embodiment.

Although the drawings represent some possible examples, the drawings are not necessarily to scale, and certain features may be exaggerated, removed, or partially sectioned to better illustrate and explain the present disclosure.

### DETAILED DESCRIPTION

Referring now to the discussion that follows and to the drawings, illustrative examples are shown and described in detail. The descriptions set forth herein are not intended to be exhaustive or otherwise limit or restrict the claims to the precise forms and configurations shown in the drawings and disclosed in the following detailed description.

Current robotic catheter control systems have not been able to help with improving the driving experience because they are typically built on the assumption that the catheter has no embedded sensor. With the introduction of an electromagnetic catheter and other electromagnetic sensor enabled devices, this barrier is surmountable, but electromagnetic technology alone is not enough to fully realize intuitive device manipulation because physicians still rely on 2D fluoroscopic images to manipulate catheters in 3D space. In certain implementations, one potential solution is to use a 3D haptic feedback input device and a 3D model of the patient's anatomy to guide the device in 3D. While this is a desirable solution, the 3D model must be accurate and adjustable because it needs to evolve as the patient anatomy changes during a procedure. In addition, the 3D model must be accurately registered to the device before it can become truly useful. Accordingly, an alternative solution is still desirable.

In this disclosure, certain implementations are provided to enhance instinctiveness in device manipulation. For example, in various implementations provided herein, device motion may be restricted to in-plane bending, if needed, to better match what is shown on the screen and what control is available on the input device. Device manipulation may be more instinctive under this control scheme than other potential solutions when traditional button controls and 2D fluoroscopic images are employed for catheterization.

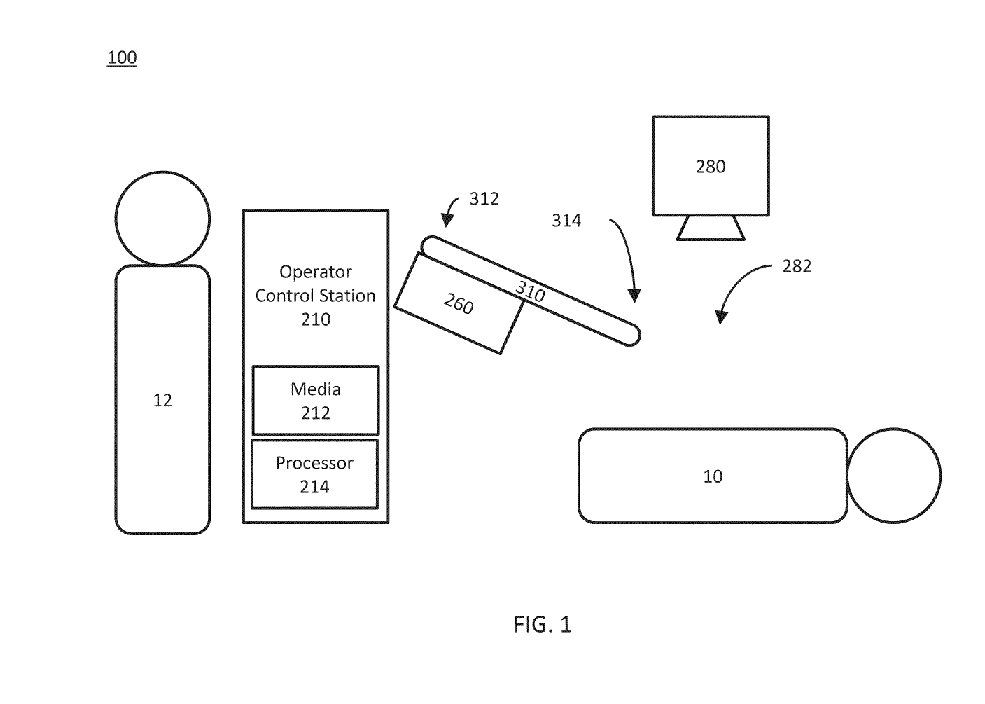

FIG. 1 illustrates a system according to certain implementations. The system 100 may include a subject 10, an operator 12, an operator control station 210, a processor 214, a controller 260, an imaging device 280, a scene of interest 282, and a device 310. The subject 10 may be a human or animal patient, or another target of a procedure. The operator 12 may be a doctor, nurse, healthcare professional, person, or artificial intelligence capable of operating the system 100 to achieve a desired result.

The operator control station 210 is a device or system usable by the operator 12 to control various aspects of the system 100, including but not limited to controlling the imaging device 280 and/or the articulation of the device 310. The operator control station 210 may include a non-transitory computer readable media 212 operably connected to a processor 214.

The processor 214 may be formed of electronic circuitry capable of carrying out operations specified by instructions, such as a computer central processing unit. The media 212 may be one or more computer-readable media operably coupled to the processor. The media 212 may take the form of transitory or non-transitory computer readable storage or memory, such as hard disk drives, solid-state storage, flash memory, network attached storage, optical storage, and/or other storage means. The media 212 may be encoded with or otherwise comprise various data and modules, such as instructions executable by the processor 214 to produce various results, including sending or receiving signals from the controller 260 relating to the articulation of the device 310, processing image data, or other functions. The media 212 may include instructions for controlling or articulating various devices or peripherals of the system 100 (e.g., the controller 260, the device 310, and the imaging device 280) according to the various methods, systems, implementations, and embodiments described herein.

The device 310 may be an articulable or manipulatable robotic catheter system or other articulable device for use in a medical or other procedure. The device 310 may include various components or tools in order to facilitate treatment or examination (e.g., a balloon for expanding a stent in an artery). The device 310 may have a proximal region 312 and a distal region 314. The proximal region 312 may be the portion or region of the device 310 coupled with the controller 260. It is the portion or region of the device 310 that remains external to, or is closest to the exterior of, the patient when the device 310 is inserted into a lumen of the subject 10. The distal region 314 may be a region opposite the proximal region 312 and may be designed to be inserted into the anatomy of the subject 10 and toward the scene of interest 282. The scene of interest 282 may be described as a location of interest or importance to a procedure. For example, the scene of interest 282 may be a portion of the anatomy of the subject 10 where a procedure will be conducted, such as a region surrounding a blocked artery. The device 310 may be a robotic device that allows the operator 12 to control the shape of the catheter. The device 310 need not be pre-shaped like typical manual catheters and may allow the device 310 to be shaped and reshaped while within the anatomy of the subject 10.

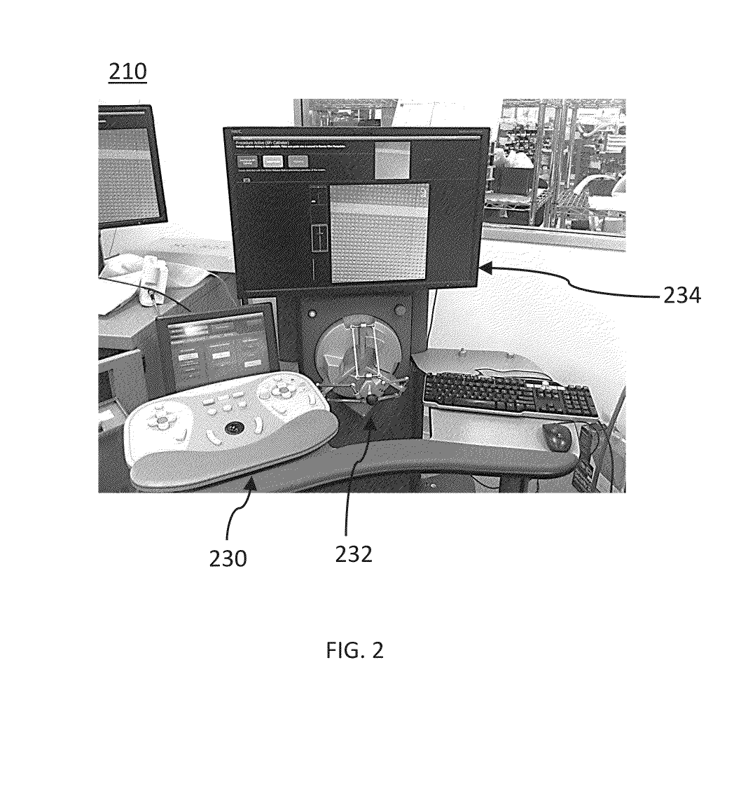

The controller 260 may be a system or a combination of systems for controlling various aspects of the system 100, including the actuation and articulation of the device 310. The controller 260 may be operably coupled to the operator control station 210 so that communication may be passed therebetween. In some embodiments, the operator 12 may directly enter commands into the controller 260 itself to control the device 310. The controller 260 may include a left-articulation user input member and a right-articulation user input member. For example, these members may be controls like those found as part of the input device 230 (see, e.g., FIG. 2) or may include any other suitable means of receiving input signals (e.g., one or more levers, joysticks, buttons, toggles, keys, etc.).

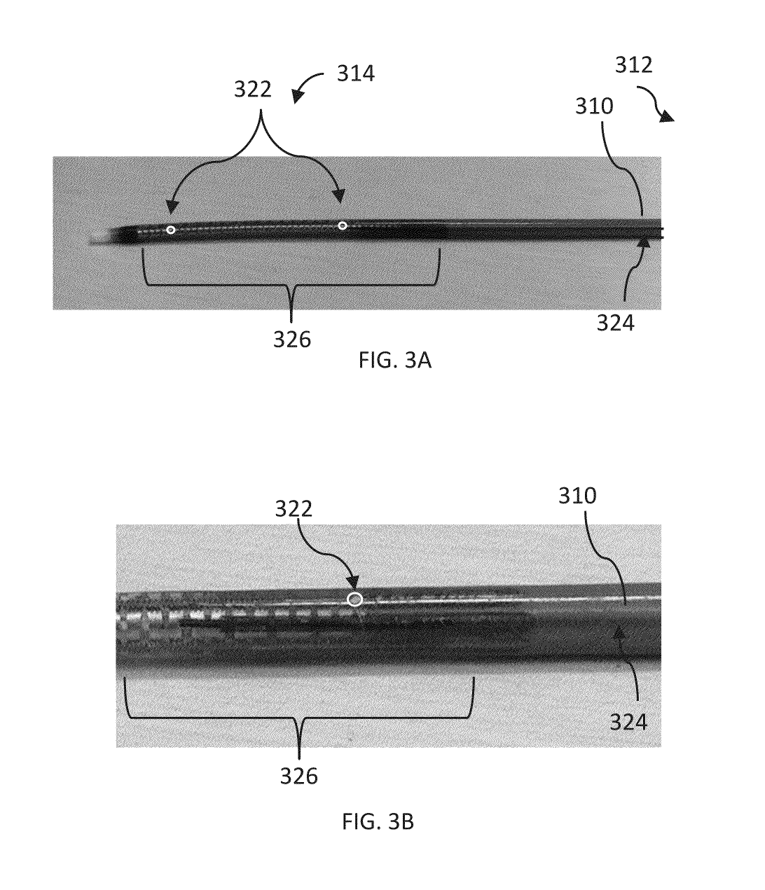

The articulation direction may generally be a particular or specified direction of distal bending. This may include, for example, a direction within full range of the locations reachable by a portion of the device 310, such as a left direction or a right direction. The controller 260 may be attached to or included within a portion of the device 310 such that the controller 260 at least partially controls or directs the motion or articulation of the device 310. For example, the proximal end 312 of a device 310 may include one or more pullwires 324, as shown in FIG. 3A, that are connectable to actuators or other machinery located within the controller 260. The actuators may be configured to push, pull, or rotate various portions of the device 310 in order to achieve particular results. As one non-limiting example, the actuators may include pulleys configured to push or pull the pullwires 324 of the device 310. The controller 260 may also include various sensors and means for providing feedback to the operator control station 210 regarding the status of the various components of the system 100.

The imaging device 280 may be a device or system of devices capable of producing 2D or 3D images or other representations of the scene of interest 282. For example, the imaging device 280 may include a device for creating images or video using radiography, magnetic resonance imaging, nuclear medicine, ultrasound, visible light, and other sources of imaging. The imaging device 280 may also include other components such as receivers or sensors for detecting the location of various medical devices, including particular portions of the device 310. The imaging device 280 may include or be connected to systems configured to process the images, for example, to improve visibility or to combine various images to create an improved representation of a particular scene of interest. The imaging device 280 may also include its own means for displaying the image or may be configured to transmit the image for display at the operator control station 210.

FIG. 2 illustrates a view of an operator control station 210 in certain implementations, including an input device 230, a 3D controller 232, and a video display 234. The operator control station 210 may include various input and output means for use by an operator 12 to send signals to the controller 260 to articulate the device 310.

The input device 230 may include various controls to command the device 310 to perform certain actions. Some such actions may include bending, rolling, advancing, deploying, retracting, and inserting the device 310 or portions thereof. The controls may include left and right bend buttons, insert and retract buttons, and a roll knob. These controls may be laid out in various arrangements including a flat or an ergonomically curved arrangement.

The video display 234 may show or display various information or controls relating to a particular operation. For example, the video display 234 may display device command input buttons, a user interface, and images or representations received from various imaging sources, for example, imaging device 280. The images or representations may include a view of the scene of interest 282 from the perspective of the imaging device 280. In some embodiments, the video display 234 includes a touchscreen configured to receive input commands directly via the screen.

FIGS. 3A and 3B illustrate a side view of one non-limiting example implementation of the distal end 314 of the device 310, including one or more sensors 322, a pullwire 324, and an articulation section 326. FIG. 3B illustrates an enlarged view of a portion of the distal end 314 of the device 310. In certain implementations, the device 310 may be an electromagnetic device, which has a set of embedded coil sensors for detecting position and orientation of the device 310.

The device 310 may be articulable and include various means for having its orientation, rotation, bending, and other articulation controlled, for example, via the pullwire 324. The pullwire 324 may be a portion of the device 310 that can be manipulated in order to control the motion or articulation of the device 310. For example, pulling on a particular pullwire 324 or combination of pullwires 324 may have a particular effect on the articulation of the device 310 within three dimensions and/or six-degrees of freedom. In certain implementations, the pullwires may be connectable at a proximal end of the device 310 to an actuator or other machinery, such as components of the controller 260, for example one or more pulleys in one or more splayers. In addition to or instead of the pullwire 324, other articulation systems may be used. The device 310 may have features that make it describable as a robotic system. For example, the device 310 may be controllable through an intermediary device rather than directly by the operator 12.

While the entire device 310 may be articulable or manipulatable, the device 310 may include an articulation section 326 that is specially or extra articulable compared to the rest of the device 310. In some embodiments, the articulation section 326 is more flexible than the remainder of the device 310. In certain implementations, the articulation section 326 may also be the only articulable section and/or a region of increased dexterity or capability.

Various portions of the device 310, such as the distal portion 314, may include one or more sensors 322 for navigation and to detect the particular positioning of the device 310 including but not limited to position of the device 310 in space, position of the device 310 in relation to a particular landmark, amount of bend in the device 310, amount of twist in the device 310, articulation amount, and other characteristics of the device 310. The sensors 322 may be, for example, electromagnetic sensors, fiber optic sensors, sensors that cooperate with external sensor systems, imaging device 280, sensors utilized in impedance-based position measurement systems, and/or other sensors.

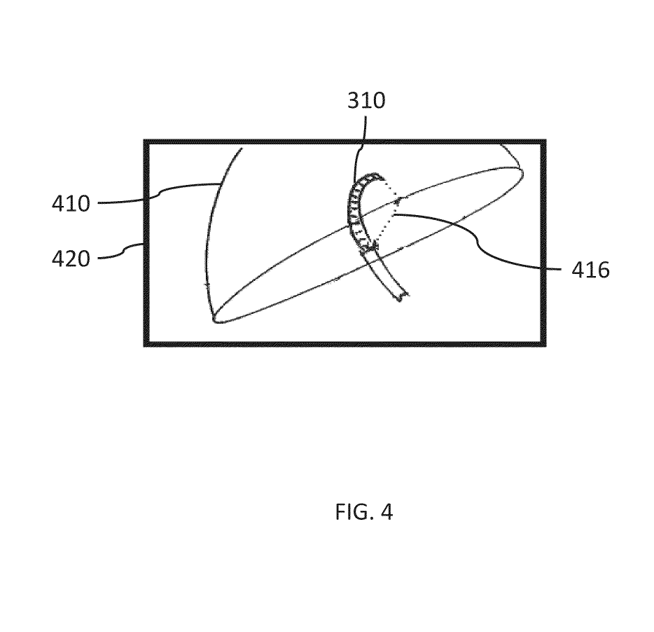

FIG. 4 illustrates a view of a device 310 from the perspective of the imaging device 280 according to certain implementations, including an image perspective plane 420, an articulation plane 416, a dome 410, and the device 310. In certain implementations, the imaging device 280 converts a 3D scene of interest 282 (e.g., internal anatomy, not shown) into a 2D representation having a particular image perspective plane 420. The articulation plane 416 may be defined by the direction of articulation or bend of the device 310 or as the current direction of articulation of the device 310, for example, the plane defined by the articulation of a catheter. In certain implementations, if the articulation plane 416 is rotated 360-degrees around the device 310, that is equivalent to a rotation of the device. The articulation plane 416 may be, for example, the only plane of bending available to the device 310 (e.g., because of a restricted range of motion), a preferred articulation plane 416, a currently defined articulation plane, one plane of many planes, and/or other range of motion. The dome 410 represents the reachable workspace for the tip of the device 310.

The above-mentioned view of the device 310 may be utilized by the operator 12 in order to perform medical treatments, such as the treatment of peripheral vascular disease. During a treatment, the operator 12 may need to navigate the device 310 through complex anatomy of the subject 10. During navigation, the device 310 may undergo significant torsional deformation as the device 310 moves through tortuous anatomy. In addition, to achieve precise navigation through the anatomy, the operator 12 may reference 2D images of 3D positioning information to receive feedback regarding the control of the device 310. The lack of additional depth information, combined with the torsional deformation of the device 310 puts a burden on the operator 12 to maintain a mental map between what the controller 260 tries to do and what the device 310 actually does. Therefore, knowing the orientation of the device 310 is a factor in building an instinctive controller. Certain implementations provided herein restrict device 310 articulation to a particular plane in order to align the expectations of the operator 12 with the actual positioning of the device 310. With such information, the controller 260 may know which pullwire 324 to pull to articulate the device 310 in the desired or user-commanded direction, regardless of which way it faces. While this method may remove one degree of freedom, the desired articulation of the device 310 is achieved more quickly and intuitively.

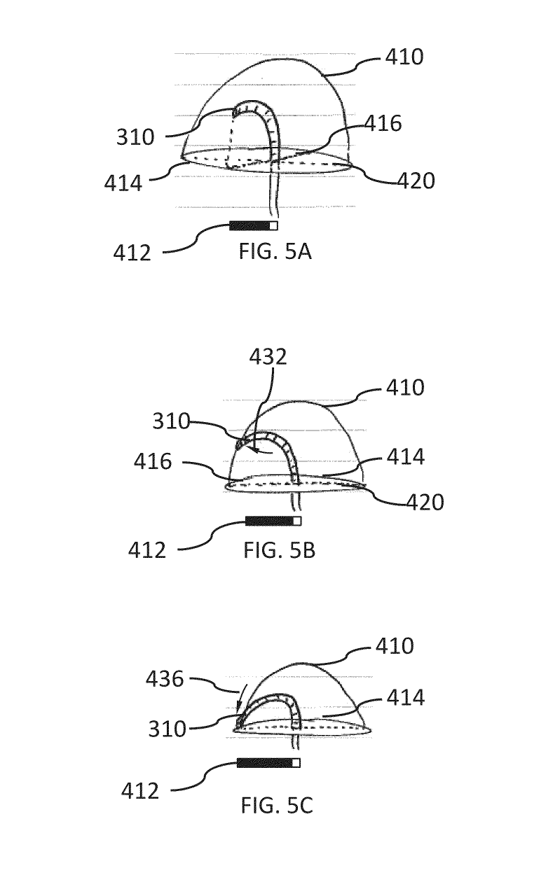

FIGS. 5A-C and 6A-6C illustrate certain implementations where an articulation plane 416 of the device 310 is in a particular relationship to the displayed image perspective plane 420 (i.e., the viewing plane), including the dome 410, the device 310, a roll plane 414, and a solid bar 412. For example, a left button on the depicted input device 230 of FIG. 2 would bend the device 310 to the left as seen on the video display 234 and a right button would bend the device 310 to the right as seen on the video display 234. The solid bar 412 can vary in length to illustrate the relative amount of control effort needed to bring the device 310 into each configuration (e.g., commanded articulation angle). The roll plane 414 is the plane on or about which the device 310 may roll. In certain embodiments, the circular, illustrated roll plane 414 shows the outer points that the device 310 may reach as the device 310 is rotated 360-degrees while in a fully articulated state. Throughout the figures, a dotted line is used to represent the articulation plane 416, a dashed line shows a portion of the figure that is parallel to the image perspective plane 420, and a line that is both dotted and dashed is used to show that the planes 416, 420 are parallel to each other. These representations of the planes 416, 420 may be referred to simply by the planes that they represent.

FIG. 5A illustrates an initial configuration of the device 310 where the articulation plane 416 is rotated about 45-degrees around the shaft of the device 310 relative to the image perspective plane 420. The device 310 is bent in a direction parallel to the articulation plane 416, which intersects the image perspective plane 420 in a particular relationship.

In certain implementations, a left bend command (e.g., as sent by the operator 12 by pressing a left bend button on the input panel 230) would further bend the device 310 in the current articulation plane 416. While this may be desirable in certain circumstances, this default may result in a less than intuitive operator 12 experience because, for example, “left”, as viewed by the operator 12 on the video display 234 may not necessarily intuitively relate to “left” in the articulation plane 416. Instead, certain implementations would, first, automatically roll the device 310 until the articulation plane 416 is parallel to the image perspective plane 420 and next start articulating in that plane. For example, as shown in FIGS. 5B-5C.

FIG. 5B illustrates an example position after the controller 260 acts on a left bend command. As shown by direction arrow 432, the device 310 is rolled until the line representing the articulation plane 416 is substantially parallel to the image perspective plane 420. The solid bar 412 has substantially the same length as in FIG. 5A, indicating that control effort needed to rotate the device 310 into the illustrated position is substantially equal to that shown in the position in FIG. 5A.

FIG. 5C illustrates an example position sometime after the controller 260 acts on another or a continued left bend command after the device 310 reached the position shown in FIG. 5B. The device 310 has bent in a direction substantially parallel to both the articulation plane 416 and the image perspective plane 420. The solid bar 412 has extended even further left than in FIGS. 5A and 5B, indicating an increase in required control effort to bring device 310 into the illustrated position.

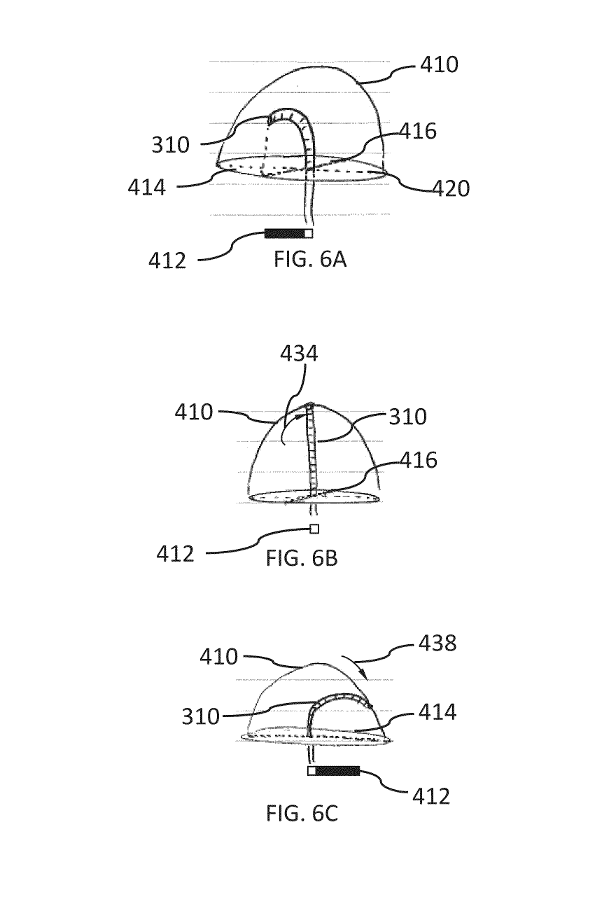

FIG. 6A illustrates the device 310 in substantially the same position as FIG. 5A. In certain implementations, while the device 310 is in this position, pressing a right bend button on the input device 230 may be selectively interpreted by the system 100 to be equivalent to pressing a relax button until the device 310 is substantially straight. Continued acting on a bend-right signal may cause the device 310 to bend away in the current articulation plane 416. While the initial straightening part is the same, once the device 310 has substantially no bend in it anymore the articulation plane 416 may instantly or automatically (e.g., without additional user input) rotate to the desired orientation (e.g., parallel to the image plane 420), and further motion of the device 310 may be restricted to the parallel plane. In certain other implementations, these steps can be reversed. For example, the articulation plane 416 is rotated to match the image perspective plane 420 and then the straightening is performed.

FIG. 6B illustrates the device 310 of FIG. 6A undergoing a “bend right” operation. Specifically, the device 310 relaxes in the direction of the arrow 434 until it is substantially straight. As can be seen, the solid bar 412 has substantially disappeared, indicating that control effort needed to bring the device 310 into the illustrated position is substantially less than the position shown in FIG. 6A. In addition, the planes 416, 420 have not been brought into alignment yet (compare FIG. 5B and FIG. 6B).

FIG. 6C illustrates the device of FIG. 6B undergoing a continued or an additional articulate right operation after the planes 416 and 420 have been aligned. Specifically, the device 310 articulates in the direction of the arrow 438. The solid bar 412 extends significantly further right than in FIG. 6B, indicating an increase in required control effort.

In certain implementations, articulating the device 310 as shown in FIGS. 5A-5C and 6A-6C, may include two steps: first, rolling the device 310 into a plane parallel to the viewing plane of the camera and, second, articulating the device 310. When the device 310 is rolled into this target plane, left and right bend buttons on the input device 230 would command the device 310 to bend left and right, respectively, as seen on the video display. Thus, observed articulation matches commanded articulation. To determine how much rotation is needed to achieve the desired roll, coordinate systems may be defined.

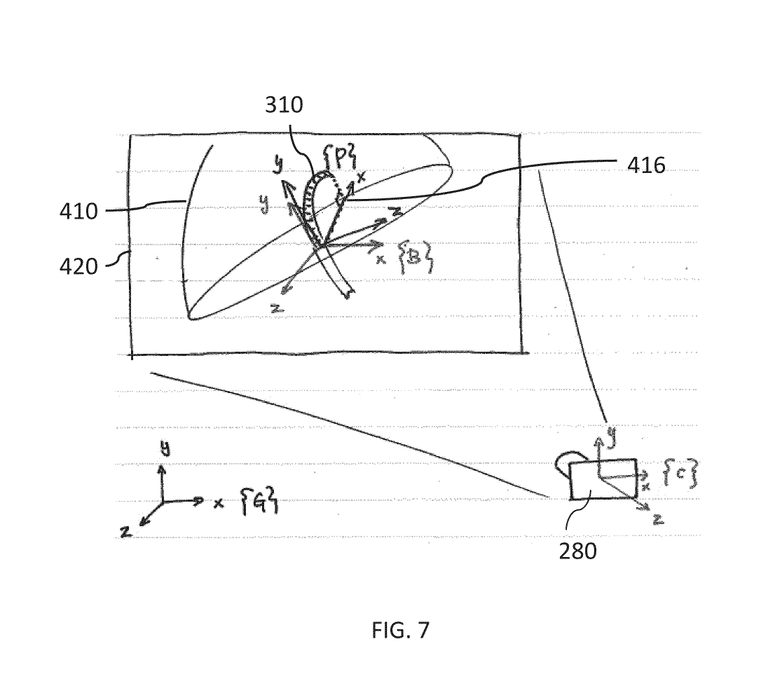

FIG. 7 illustrates a device 310 from the perspective of the imaging device 280 according to certain implementations, including a superimposed coordinate system, the image perspective plane 420, the articulation plane 416, the dome 410, and the device 310. The coordinate system may be determined, for example, by operator control station 210, processor 214, controller 260, or other part or parts of the system 100. The coordinate system may be determined based on, for example, measured sensor data from the device 310, imaging device 280, or other part or parts of the system 100. With respect to the coordinate system, {B} denotes the frame attached to the base of the articulation section 326 and {P} is the frame attached to the articulation plane 416. {B} and {P} initially overlap when there is no roll, but {P} separates from {B} as the device 310 starts to roll (e.g., {P} rotating around its y axis). {C} represents the frame of the image perspective plane 420. The image perspective plane 420 may be described as a viewing point and the virtual world is rendered from this particular location. {G} defines the global coordinate system for the world. In addition, x_{c} and y_{c} represent what may be referred to as that which is right and up, respectively, as seen from the imaging device 280. If the input device 230 controls were instinctive, a bend right command would bend the device 310 toward the positive x_{c} direction, and a bend left command would bend the device 310 toward the negative x_{c} direction.

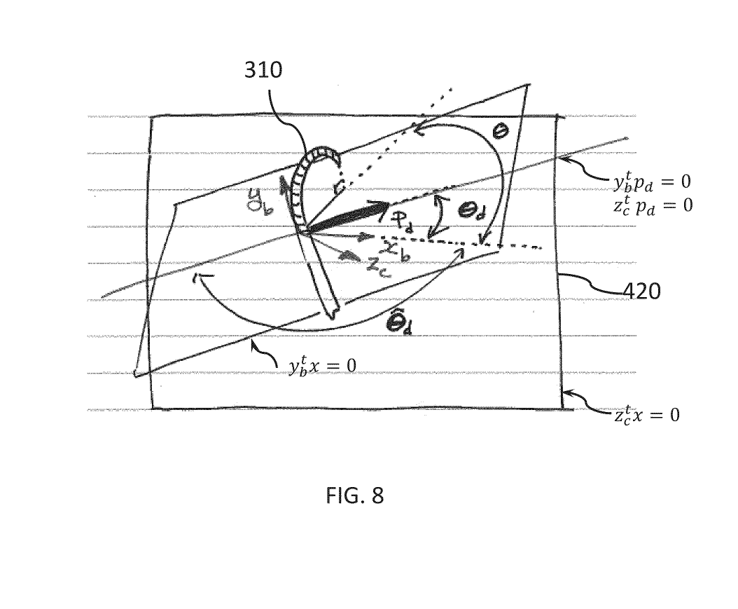

With reference to FIG. 8, assuming that all calculations are done in {B}, the idea is to find a vector, p_{d}, such that it is embedded in the line defined by the two planes perpendicular to y_{b} and z_{c}, respectively. Both planes go through the origin of {B}. The first of the two is the x_{b}-z_{b} plane in {B}

y_{b}^{T}x=0 [1]

and the second is the x_{c}-y_{c} plane in {C} shifted in negative z_{c} direction to go through the origin of {B}

Z_{c}^{T}x=0 [2]

where Z_{c} is measured in frame {B}, i.e., B_{Z}_{_{c}}. The pre-superscript denotes the coordinate system the vector is defined in. This can be rewritten as the following:

$$\begin{matrix}{B}_{{z}_{c}} = {}_{C}^{B}{R}{}_{}^{C} {z}_{c} \\ = {( {}_{C}^{B} R )}^{T} {z}_{c} {}_{C}^{G}{R}{}_{}^{C} {z}_{c}\end{matrix} \qquad [ 3 ]$$

where _{B}^{G}R is an orientation measurement from a sensor in the device 310 and _{C}^{G}R is the camera orientation. C_{Z}_{_{c}} is simply the z vector in its own coordinate system, i.e., (0 0 1)^{T}. The intersection of the two planes forms a line, l, that passes through the origin of {B}. By construction, line l is perpendicular to z_{c} (e.g., embedded in the plane parallel to the image perspective plane 420, but shifted in z_{c} direction to pass through the origin of {B}), and at the same time perpendicular to y_{b}, which makes it possible to measure its roll as the angle measured around y_{b} between x_{b} and line l.

Assume that x=p_{d} satisfies Equation [1] and Equation [2]. Equation [1] essentially says p_{d}_{_{y}} is zero because p_{d} is perpendicular to y_{b}. With p_{d}_{_{y}} set to zero, Equation [2] gives a fixed ratio of p_{d}_{ₓ} to p_{d}_{_{z}} as shown in the following:

$${p}_{{d}_{z}} = - \frac{{z}_{{c}_{x}}}{{z}_{{c}_{z}}} {p}_{{d}_{x}} \qquad [ 4 ]$$

Any combination of p_{d}_{ₓ} and p_{d}_{_{z}} that satisfies Equation [4] would be on the line l. If, for example, p_{d}_{ₓ} is arbitrarily chosen to be one, then p_{d} can be rewritten as the following:

$${p}_{d} = [ \begin{matrix}1 \\ 0 \\ - \frac{{z}_{{c}_{x}}}{{z}_{{c}_{z}}}\end{matrix} ] \qquad [ 5 ]$$

where z_{c}_{ₓ} and z_{c}_{_{z}} are the first and third elements in B_{Z}_{_{c}}. Note that these are not (1 0 0)^{T} and (0 0 1)^{T} since they are vectors in {B}. Now that p_{d} is known, the desired roll angle can be calculated as the following:

$$\begin{matrix}{θ}_{d} = arctan ( - \frac{{pd}_{z}}{{pd}_{x}} ) \\ = arctan ( - \frac{{z}_{{c}_{x}}}{{z}_{{c}_{z}}} )\end{matrix} \qquad [ 6 ]$$

There is a complementary desired roll angle, \hat{θ}_{d} that would also roll the articulation plane 416 into the desired orientation, although the plane normal would be flipped as shown.

\hat{θ}_{d}=θ_{d}−π [7]

Of the two desired roll angles, θ_{d} and \hat{θ}_{d}, the controller 260 may determine which angle to use. One way the controller may make the decision is to base it on their respective magnitude, such that the one closer to the current roll angle, θ, is chosen.

It is believed that the proposed method would simplify device 310 driving, yet make it more intuitive and instinctive, especially for operators 12 that are still new to the driving mechanics of a robotic device system.

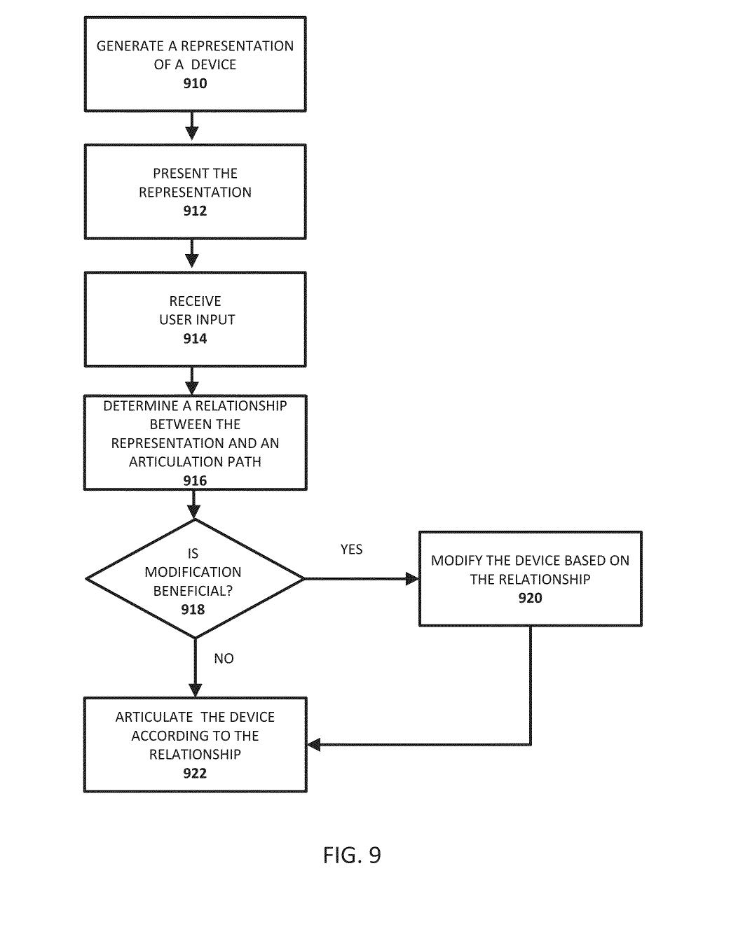

FIG. 9 illustrates a method for using the above system 100 in order to articulate a device 310. At step 910, a representation 236 of a device 310 is generated. For example, the imaging device 280 generates a direct fluoroscopic representation 236 of a device 310 within the anatomy of the subject 10. The representation 236 may be a 2D representation of a 3D scene of interest 282. The representation 236 may be a description of the device 310 generated by the imaging device 280 or other sensor. The representation 236 may be a direct image of a scene of interest 282 that includes the device 310; however, the representation 236 need not be a direct representation. For example, the representation 236 may be a composite of several different sources of information, an artificial representation of the device 310 based on a source of information, and other indirect methods of representing data regarding the device 310 or scene of interest 282. While the representation 236 may typically be an image, it need not be. The representation 236 may be a collection of data regarding the device 310, for example as may be used by a computer in a decision making process.

At step 912, the representation 236 is presented. For example, the fluoroscopic representation of the device 310 within the anatomy of the subject 10 is presented to the operator 12 at the monitor 234 of the operator control station 210. This step 912 may include providing the representation 236 to another entity or part of a process. The representation 236, either alone or in the way it is presented, may include certain specific characteristics. For example, if the representation 236 is a 2D representation of a 3D scene of interest 282 (or a 3D image presented on a 2D monitor), the representation may include certain indicia of depth and be restricted in scope to a particular plane.

At step 914, the system 100 receives user input. For example, the operator 12 may, based on the representation 236, push a button on the input panel 230 to direct the controller 260 to bend the distal tip of the device 310 to the right (or in another desired direction). The input may be generally an instruction regarding how the device 310 should be operated, such as a direction to articulate the device 310 in a particular manner. The input may be received from a variety of sources, including but not limited to buttons, knobs, dials, switches, touchscreens, other hardware (e.g. levers, joysticks, sliders), software processes, networked devices, and other potential sources of communication.

At step 916, the system 100 determines a relationship between the representation and the articulation plane. For example, the system 100 may detect that the articulation plane of the device 310 is offset 45-degrees (or any other detectable angle) from the plane of the direct fluoroscopic representation of the device 310. In certain implementations, the relationship is between the articulation plane and the characteristic of the representation. For example, the relationship may be the angle between the plane of the representation and the plane of the articulation plane. As another example, the relationship may be an offset distance in space, such as a distance between a perceived location of the device 310 and an actual location. The articulation plane may be an articulation plane 416 as described and/or determined above. The relationship, or factors relating thereto, may be detected by the use of the sensors 322 on the catheter 314, the imaging device 280, image recognition software, and other sensors or sources. This relationship may be used to determine an amount of modification or adjustment for adjusting the device 310.

At step 918, the system 100 determines whether it is beneficial to first modify the orientation of the device 310 based on the relationship or to simply articulate the device 310 according to the modified relationship. For example, the system 100 may determine whether the image perspective plane and articulation plane are already substantially parallel or whether one of the planes requires rotation in order for the planes to be substantially parallel. The system 100 may additionally or alternatively determine whether modification of the orientation of the device 310 would result in a more intuitive device manipulation experience. In one non-limiting example, the system 100 may determine that the device 310 has already been rolled into just the right anterior-posterior orientation so that there is no need to roll the device 310 any further; in such an example, the system 100 may decide to skip step 920 and proceed directly to step 922. In certain implementations, the system 100 may lock a particular movement of the device (e.g., the roll angle of the articulation plane 416 of the device 310 may be locked rather than manipulatable). In certain embodiments, the determination of benefit, intuitiveness, and/or whether the planes are substantially parallel may be based on various factors including a heuristic determination of intuitiveness, a set preferences, whether the relationship exceeds a predetermined threshold (e.g., an angle of difference, such as a 1, 5, 10, or 20 degree difference), and other factors or combinations of factors.

At step 920, the device 310 is modified based on the relationship. For example, the device 310 may be rotated until the articulation plane 416 of the device 310 is substantially parallel to the plane of the direct fluoroscopic representation of the device 310 (e.g., image perspective plane 420). In certain implementations, as the articulation plane 416 is adjusted to converge to the image perspective plane 420, all articulation becomes visible on the display 234. Modifying the device 310 may include articulating or rotating the device 310 or changing it in some way to take into account the relationship.

At step 922, the device 310 is articulated according to the modified relationship. For example, the device 310 may be bent in a direction toward the left or other direction within or substantially parallel to the image perspective plane 420 (as seen on the display 234). This step may be as simple as executing the instruction received in step 914 above.

Certain implementations have been presented, which may facilitate intuitive or instinctive device manipulation, including systems for use with a planar input device such as an input device 230 and a planar feedback device such as a display 234. In certain implementations, instead of augmenting the inherently 2D user interface to enable 3D device driving, conscious efforts have been made to restrict device driving to a 2D plane such that the resulting motion is easily identifiable on the display 234.

From the foregoing it will be appreciated that, although certain implementations or embodiments have been described herein for purposes of illustration, various modifications may be made without deviating from the spirit and scope of the disclosure. For example, an articulation command, in some implementations, may cause the device 310 to instantly or automatically rotate so the articulation plane 416 and image perspective planes 420 are parallel, while in other implementations, the command may act as a kind of “rotate” command until the planes 416, 420 are substantially parallel and then begin articulating the device 310 in the desired direction. The described features may be implemented in a toggleable fashion such that the operator 12 may toggle when the mode is on or off. In addition, the various characteristics or preferences may be saved as a setting in the media 212 such that, for example, an operator 12 may have a certain profile within the system 100 that may be selected to load the preferences of the operator 12. This may facilitate the use of the system by multiple operators 12, each having different preferences.

The mechanisms and methods described herein have broad applications. The foregoing embodiments were chosen and described in order to illustrate principles of the methods and apparatuses, as well as some practical applications. The preceding description enables others skilled in the art to use the methods and apparatuses in various embodiments and with various modifications, as suited to the particular use contemplated. In accordance with the provisions of the patent statutes, the principles and modes of operation of this disclosure have been explained and illustrated in exemplary and preferred embodiments.

This disclosure may be practiced differently than is specifically explained and illustrated without departing from its spirit or scope. Various alternatives to the embodiments described herein may be employed in practicing the claims without departing from the spirit and scope as defined in the claims. The scope of the disclosure should be determined, not with reference to the above description, but should instead be determined with reference to the appended claims, along with the full scope of equivalents to which such claims are entitled. Future developments may occur in the arts discussed herein, and the disclosed systems and methods may be incorporated into such future examples. Furthermore, all terms used in the claims are intended to be given their broadest reasonable constructions and their ordinary meanings as understood by those skilled in the art unless an explicit indication to the contrary is made herein. In particular, use of singular articles such as “a,” “the,” “said,” etc. should be read to recite one or more of the indicated elements unless a claim recites an explicit limitation to the contrary. At times, the claims and disclosure may include terms such as “a plurality,” “one or more,” or “at least one;” however, the absence of such terms is not intended to mean, and should not be interpreted to mean, that a plurality is not conceived.

As used herein, the term “comprising” or “comprises” is intended to mean that the device, system, or method includes the recited elements, and may additionally include any other elements. “Consisting essentially of” shall mean that the device, system, or method includes the recited elements and excludes other elements of essential significance to the combination for the stated purpose. Thus, a device, system, or method consisting essentially of the elements as defined herein would not exclude other elements that do not materially affect the basic and novel characteristic(s) of the claimed invention. “Consisting of” shall mean that the device, system, or method includes the recited elements and excludes anything more than trivial or inconsequential elements. Embodiments defined by each of these transitional terms are within the scope of this disclosure.

Accordingly, the invention or inventions included herein are limited only by the following claims.

1. A method for manipulating a catheter within a lumen of a body, the method comprising:
providing a manipulatable catheter system, comprising:
a catheter having a proximal end, a distal end, an articulable distal portion, and a sensor disposed along the distal portion, and
a controller coupled with the catheter proximal end, wherein the controller controls articulation of the distal portion of the catheter;
displaying an image of at least the distal portion of the catheter on a video display;
receiving, via the controller, a user input directing the distal portion of the catheter to articulate in an articulation direction;
determining a relationship between an articulation plane of the distal portion of the catheter and a viewing plane of the image on the video display;
automatically adjusting the catheter, using the controller, to move the articulation plane of the distal portion of the catheter closer to parallel with the viewing plane of the image, based on the determined relationship; and
articulating the distal portion of the catheter in the articulation direction, using the controller, based on the user input.

2. The method of claim 1, wherein determining the relationship comprises determining a difference in orientation between the articulation plane and the viewing plane.

3. The method of claim 1, wherein adjusting the catheter comprises aligning the articulation plane with the viewing plane so that the two planes are parallel.

4. The method of claim 1, wherein adjusting the catheter comprises rotating the catheter.

5. The method of claim 1, wherein adjusting the catheter comprises adjusting the articulation direction to align the articulation plane with the viewing plane.

6. The method of claim 1, wherein the controller comprises a left-articulation user input member and a right-articulation user input member, and wherein the articulation direction comprises a left direction or a right direction.

7. The method of claim 1, wherein the image comprises a fluoroscopic image.

8. The method of claim 1, further comprising determining an amount of adjustment before adjusting the catheter.

9. The method of claim 8, wherein determining the amount of adjustment comprises:
determining a coordinate system;
determining the viewing plane within the coordinate system; and
determining the articulation plane within the coordinate system.

10. The method of claim 1, further comprising:
receiving an additional user input instructing the controller to allow the distal portion to articulate in the articulation plane without adjusting the catheter; and
articulating the distal portion of the catheter as instructed by the additional user input.

11. A method for manipulating a catheter within a lumen of a body, the method comprising:
providing a manipulatable catheter system, comprising:
a catheter having a proximal end, a distal end, an articulable distal portion, and a sensor disposed along the distal portion, and
a controller coupled with the catheter proximal end, wherein the controller controls articulation of the distal portion of the catheter;
displaying a representation of at least the distal portion of the catheter on a video display that defines a viewing plane;
receiving, via the controller, a user input directing the distal portion of the catheter to articulate in a left direction or a right direction, relative to the representation on the video display;
automatically adjusting the catheter to align an articulation plane of the distal portion of the catheter with viewing plane; and
articulating the distal portion of the catheter to the left or right, according to the user input, within the articulation plane.

12. The method of claim 11, wherein adjusting the catheter comprises aligning the articulation plane with the viewing plane so that the two planes are parallel.

13. The method of claim 11, wherein adjusting the catheter comprises rotating the catheter.

14. The method of claim 11, wherein the image comprises a fluoroscopic image.

15. The method of claim 11, further comprising determining an amount of adjustment before adjusting the catheter.

16. The method of claim 15, wherein determining the amount of adjustment comprises:
determining a coordinate system;
determining the viewing plane within the coordinate system; and
determining the articulation plane within the coordinate system.

17. A system for manipulating a catheter within a lumen of a human or animal subject, the system comprising:
a catheter having a proximal end, a distal end, an articulable distal portion configured to articulate in three dimensions, and a sensor disposed along the distal portion;
a controller coupled with the catheter proximal end to bend the distal portion; and
a processor coupled with the controller and configured to execute instructions to perform a method, comprising:
determining a coordinate system;
determining a viewing plane within the coordinate system, wherein the viewing plane is defined by an image of the distal portion of the catheter on a video display;
determining an articulation plane of the distal end of the catheter;
receiving a user input directing the distal portion of the catheter to articulate in a direction; and
automatically adjusting the catheter to align the articulation plane with the viewing plane.

18. The system of claim 17, wherein the user input comprises an instruction to articulate the distal portion of the catheter in a left direction or a right direction.

19. The system of claim 17, wherein the processor is further configured to generate an articulation signal to cause an actuator to articulate the distal portion of the catheter according to the user input.

20. The system of claim 17, wherein adjusting the catheter comprises rotating the catheter so that the articulation plane is parallel with the viewing plane.
